(19) United States
(12) Patent Application Publication
(10) Pub. No.: US 2007/0167844 A1
(43) Pub. Date: Jul. 19, 2007

(54) Apparatus and method for blood pressure measurement by touch

(76) Inventors: Haruhiko H. Asada, Lincoln, MA (US); Aleksandar Marinkovic, Boston, MA (US); Andrew T. Reisner, Newton, MA (US); Phillip Shaltis, Newton, MA (US)

Correspondence Address: BROMBERG & SUNSTEIN LLP, 125 SUMMER STREET, BOSTON, MA, 02110-1618, US

(21) Appl. No.: 11/605,935

(22) Filed: Nov. 29, 2006

Related U.S. Application Data
(60) Provisional application No. 60/740,375, filed on Nov. 29, 2005.

(51) Int. Cl. A61B 5/02; A61B 5/103

(52) U.S. Cl. 600/485; 600/587; 600/490; 600/504

(57) ABSTRACT

An arterial blood pressure apparatus and methods for using the apparatus are disclosed. The apparatus has a force transfer element for applying unidirectional force to a body part of the subject and a pressure sensor for generating a signal based on the unidirectional force. Multiple degrees of force may be applied simultaneously or serially. The apparatus also has a second sensor for generating a signal associated with blood volume within a blood vessel of the body part and a processor for inferring arterial blood pressure from the force sensor signal and the second sensor. The apparatus may take the form of a glove or a wand.

The present application claims priority from US Provisional Application Ser. No. 60/740,375, filed Nov. 29, 2005, which application is incorporated herein by reference in its entirety.

### TECHNICAL FIELD

The present invention relates to methods and apparatus for measuring arterial blood pressure (ABP), and, more particularly, for measuring ABP with unidirectionally applied pressure as may be provided under extenuating pre-hospital conditions.

### BACKGROUND OF INVENTION

FIG. 1 is a computer simulation 100 showing the cuff pressure for an oscillometric device as a function of time.

At time 0 (110), the cuff is inflated to over 160 mmHg (which is greater than the systolic blood pressure) and there are no significant oscillations. As the cuff deflates to approximately 100 mmHg (time 80 seconds) (120) there appears a pressure fluctuation inside the cuff, caused by the volume change of the brachial artery opening and closing as its intra-luminal pressure crosses the external cuff pressure. The magnitude of these oscillations is maximal when the cuff pressure equals the mean intra-arterial pressure (80 mmHg) (130). As the cuff pressure falls below the mean arterial pressure, the magnitude of the oscillations falls, until the cuff pressure falls below diastolic blood pressure, and the magnitude of the oscillations no longer varies significantly as a function of external cuff pressure.

It has been suggested that medical responses during extenuating circumstances (military battle, natural disasters, etc.) should enable standard, established methods of assessing patients, grading severity, and therapeutic decision-making. Virtually all existing triage systems for sorting patients into categories of severity/priority make use of arterial blood pressure (ABP) and more specifically the systolic blood pressure. Caregivers also use ABP in their therapeutic decision-making. Without ABP, a field response system must re-invent, re-validate, and re-train caregivers in a whole new paradigm of assessing patients and making treatment decisions, rather than using familiar standard-of-care methodologies well-established over the past century. Yet existing technologies for measuring ABP are suboptimal for the field, for several reasons:

Existing ABP instrumentation requires careful placement, such as of an oscillometric cuff, which must be placed around the arm at the level of the brachial artery, or a tonometer-type device, which must be properly placed over the radial artery.

Existing ABP instrumentation is not motion tolerant.

Existing ABP instrumentation can require seconds or minutes to make a single measurement, which consumes valuable responder time and reduces the feasibility of timely re-evaluation.

Finally, and unfortunately, even in routine clinical conditions, ABP measurement is famously confounded by human error and poor technique.

These shortcomings are particularly grave since during the chaos and stress of an uncontrolled prehospital environment, when a set of vital signs can determine the fate of an individual (e.g. triage category), it is more important than ever that ABP be accurately measured.

The signal provided by a plethysmogram, such as a photoplethysmogram (PPG), is related to the underlying vascular volume pulsations. Ando et al., Pressure-volume relationships of finger arteries in healthy subjects and patients with coronary atherosclerosis measured non-invasively by photoelectric plethysmography, Jpn. Circ. J., vol. 55, pp. 567-75 (1991), incorporated herein by reference, showed that these volumetric changes are related to the patient's ABP through a nonlinear compliance relationship.

Discussions of prior art may be found in the following references, all of which are incorporated herein by reference:
i. Talke, P. O., Measurement of systolic blood pressure using pulse oximetry during helicopter flight. Crit Care Med, 1991. 19(7): p. 934-7.
ii. Movius, A. J., S. L. Bratton, and G. K. Sorensen, Use of pulse oximetry for blood pressure measurement after cardiac surgery. Arch Dis Child, 1998. 78(5): p. 457-60.
iii. McCluskey, B., et al., Out-of-hospital use of a pulse oximeter to determine systolic blood pressures. Prehospital Disaster Med, 1996. 11(2): p. 105-7.
1. Edmonds, Z. V., et al., The reliability of vital sign measurements. Ann Emerg Med, 2002. 39(3): p. 233-7.
2. Jones, D. W., et al., Measuring blood pressure accurately: new and persistent challenges. Jama, 2003. 289(8): p. 1027-30.
3. Ando, J., et al., Pressure-volume relationships of finger arteries in healthy subjects and patients with coronary atherosclerosis measured non-invasively by photoelectric plethysmography. Jpn Circ J, 1991. 55(6): p. 567-75.
4. Langewouters, G. J., et al., Pressure-diameter relationships of segments of human finger arteries. Clin Phys Physiol Meas, 1986. 7(1): p. 43-56.
5. Yamakoshi, K., Non-invasive and simultaneous cardiovascular measurements using plethysmography techniques. Front Med Biol Eng, 1990. 2(4): p. 249-76.
6. Challoner, A. V. J., Photoelectric plethysmograph, in Non-Invasive Physiological Measurements, P. Rolfe, Editor. 1979, Academic Press: London. p. 125-151.
7. Sara, C. A. and C. A. Shanks, The peripheral pulse monitor—a review of electrical plethysmography. Anaesth Intensive Care, 1978. 6(3): p. 226-33.
8. Ugnell, H. and P. A. Oberg, The time-variable photoplethysmographic signal; dependence of the heart synchronous signal on wavelength and sample volume. Med Eng Phys, 1995. 17(8): p. 571-8.
9. Asada, H. H., et al., Mobile monitoring with wearable photoplethysmographic biosensors. IEEE Eng Med Biol Mag, 2003. 22(3): p. 28-40.

### SUMMARY OF THE INVENTION

In accordance with preferred embodiments of the present invention, an apparatus is provided for measuring arterial blood pressure in a subject. The apparatus has a force transfer element for applying unidirectional force to a body part of the subject and a first sensor, such as a pressure sensor for producing a signal based on the unidirectional force. Multiple magnitudes of force may be applied simultaneously or serially. The apparatus also has a second sensor that produces a signal associated with the blood pressure within a blood vessel of the body part and a processor for inferring arterial blood pressure from the force sensor signal and the signal of the second sensor.

In certain embodiments of the invention, the pressure sensor may also serve the role of the second sensor by detecting a pulsatile component of the pressure. The second sensor may also, however, be a plethysmograph and measure changes in volume of the underlying artery. More particularly, the plethysmograph may be a photoplethysmograph. From a maximum plethysmographic signal, it may be inferred that the transmural pressure is zero and thus the arterial blood pressure equals the applied pressure. In such an embodiment, the arterial blood pressure is the mean arterial blood pressure of the subject. In other embodiments, the apparatus can measure the systolic blood pressure and diastolic blood pressure based on the force sensor signal and the signal of the second sensor. For example, the controller can determine when the artery stops oscillating and is therefore no longer pulsatile. When the artery stops oscillating in response to increasingly applied pressure, the pressure that is applied is greater than or equal to the systolic blood pressure.

The apparatus may also include a light source wherein the light source may be either a single light emitting diode (LED) or an array of LEDs.

In accordance with various alternate embodiments of the invention, the force transfer member may be a wand or a glove, and the plethysmograph may be a photoplethysmograph. The force transfer element may be characterized by a plurality of pressure regions each having a distinctive compliance.

In various embodiments, there may be a plurality of force sensors. In such embodiments, each force sensor may be coupled to a mount having a different compliance characteristic.

Further, the apparatus can include a display for providing information to the caregiver. For example, the controller may provide feedback to the caregiver through the display or sound instructing the caregiver to reposition the apparatus in order to find a strong sensor signal. The controller may also indicate through the display or sound that the caregiver should apply more or less pressure. Thus, as the caregiver applies multiple pressures, the controller receives multiple pressure and sensor signal pairs from which the arterial blood pressure can be determined.

### BRIEF DESCRIPTION OF THE DRAWINGS

The features of the invention will be more readily understood by reference to the following detailed description, taken with reference to the accompanying drawings, in which:

FIG. 1 is a computer simulation showing the cuff pressure for an oscillometric device as a function of time;

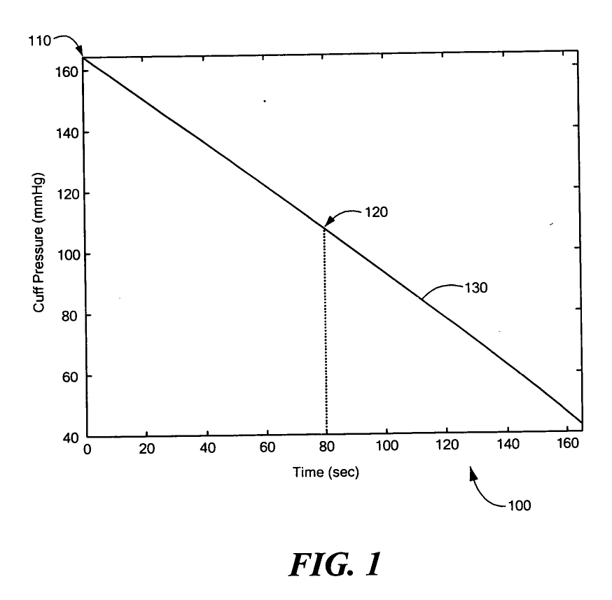

FIG. 2 shows an embodiment of the invention for measuring arterial blood pressure wherein pressure is applied through a glove;

FIG. 3 shows an embodiment of the invention for measuring arterial blood pressure wherein pressure is applied through a wand;

FIG. 4 is a flow chart teaching a methodology for obtaining arterial blood pressure using embodiments of the invention;

FIG. 5 shows a sigmoid-shaped compliance curve that demonstrates how a PPG output is a function of the pressure difference across a vascular wall;

FIG. 6 graphically depicts a PPG waveform and pressure waveform over time; and

FIG. 7 shows an example of a sensor unit configuration that includes LEDs and photo sensors.

### DETAILED DESCRIPTION OF PREFERRED EMBODIMENTS

The terms “processor” and “controller” shall be used interchangeably within the detailed description and appended claims. The term “arterial blood pressure” shall refer to both the temporal waveform of pressures exerted on the arterial walls and/or the customary clinical features including discrete measurements such as, systolic, mean, and diastolic blood pressures unless the context indicates otherwise.

The underlying principle for oscillometry is that the compliance of an arterial wall is non-linear with respect to transmural pressure (the net pressure acting across the wall). The artery is maximally compliant (i.e. demonstrates the greatest volume change for a given pressure change) when the pressure applied from the outside is equal to the internal mean arterial pressure (MAP). When the artery compliance is low, the volume oscillations caused by the pulsatile arterial blood pressure signal are relatively small. Conversely, the maximum compliance results in a maximum waveform amplitude reflected as an increased oscillation as the unloaded artery expands and contracts. This assumes that the underlying pulse pressure doesn't change much during the measurement interval, and that there is negligible motion artifact. This method is limited to intermittent use, since it does not obtain any information about the underlying compliance curve and consequently requires repeated exposure to occluding pressures for discrete blood pressure measurements.

In accordance with preferred embodiments of the invention, the external cuff of prior art methods is replaced, as actuator, by a caregiver who presses the sensor against the subject, thereby applying different pressures. The human operator replaces the inflation of the cuff, while the sensor measures the magnitude of the resultant oscillations. Therefore, no cuff or other actuator is needed. This device may be manufactured, for example, in the form of a glove or a wand.

This device is held by the operator, through an intervening wand or glove, for example, and pressed over a body part of a subject that contains an artery. It can be therefore applied to any location with a palpable arterial pulse, including over the radial artery, carotid artery, femoral artery, temporal artery, digital artery, and the pedal pulses.

The biosensor in the wand or glove can rapidly measure the arterial blood pressure (ABP). Mean arterial blood pressure may be measured by determining when maximum compliance of an artery occurs without the need for using a cuff as is necessary in traditional oscillometry.

Advantages of such a device and method may be realized in a prehospital environment, including combat casualty care, responses to a natural disaster, and responses to a civilian trauma with difficult access to the casualties (e.g. car crash with difficult extrication). It may also provide advantage over prior art devices and methods when used in conventional healthcare under circumstances where expeditious ABP measurement is valuable. The biosensor unit that is made a portion of the glove or wand is able to measure the peak of arterial blood pressure (systolic blood pressure, or SBP), by measuring the external pressure applied by the caregiver over the artery, and ascertaining when the underlying artery collapses. The oscillations of the artery can be sensed by a photo sensor and determine the transition point between a pulsatile and a non-pulsatile signal.

In a prehospital environment, the pressure sensor is positioned overlying an artery, it measures (such as by the displacement of its diaphragm) pressure caused by the underlying tissue's movement (i.e., volume change), which is in turn caused by the arterial pressure changes. But because the arterial wall and tissue itself are elastic and hold tension, the relationship between the arteries internal pressure and the tissue's volume movement and the external pressure sensor's diaphragm movement and the external pressure measured are quite complex. In the return-to-flow application, however, measurement of either volume or pressure may suffice for purposes of rendered the binary decision: whether the artery is pulsating or collapsed. Thus, the SBP can be determined with this binary decision.

Detection of the loss of arterial pulsations can be obtained using either the pressure sensor itself, or an accompanying photoplethysomgraph (PPG) sensor unit. The presence of a pulsatile PPG may indicate when the subject's systolic blood pressure (SBP) exceeds the pressure applied by the caregiver. Embodiments of the invention are especially useful if auscultation and oscillometry are not practical.

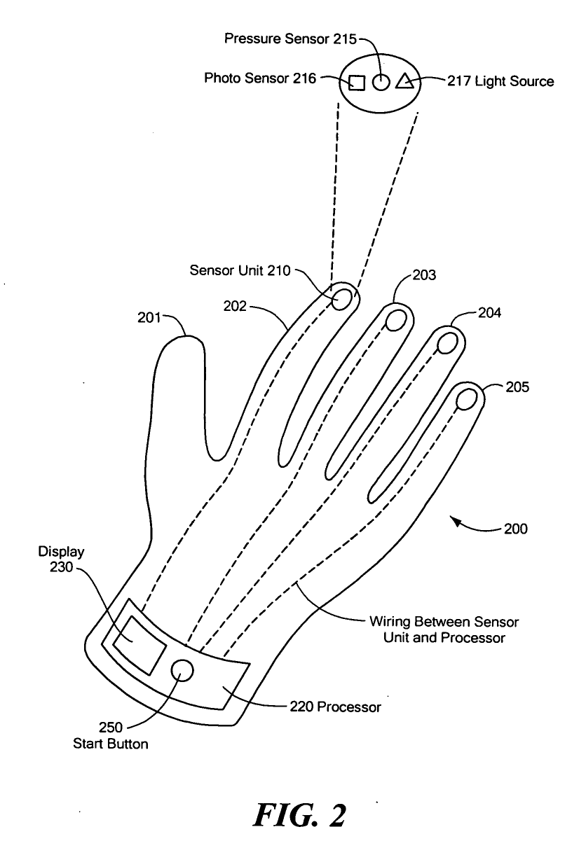

FIG. 2 shows an embodiment of the invention for measuring arterial blood pressure by applying an external pressure substantially normal to a body part of a subject. In the shown embodiment, a sensor unit 210 is provided on at least one finger 201-205 of a glove 200. The sensor unit 210 includes a pressure sensor 215 along with a photo sensor 216 and a light source 217. Different types of pressure sensors may be used with embodiments of the invention including but not limited to potentiometric, inductive, capacitive, piezoelectric, strain gauge and piesoressitve integrated semiconductor. In certain embodiments, the photo sensor 216 is a photoplethysomgraph and the light source 217 is a light emitting diode (LED). In this embodiment, a caregiver places the glove 200 on his hand and applies varying pressure to a body part above an artery. Thus, varying pressure is applied to an artery. The pressure sensor 215 that is located in at least one finger of the glove senses the applied pressures of the caregiver. As pressure is applied, the LED 217 passes light through the body part and the artery. The photo sensor 216 senses the light intensity of one or multiple wavelengths of light from the LED 217 and produces an output signal indicative of the pulsations of the artery. The output signals from the pressure sensor and the photo sensor 216 are provided to a processor 220, which may also be referred to as a controller. The processor 220 is electrically coupled to both the photo sensor 216 and the pressure sensor 215 and the processor uses known algorithms to determine when the artery has collapsed for determining the SBP of the patient. In response to either auditory or visual indicators by the processor, the caregiver applies different pressures to the body part. When the pulsatile component of the photo sensor signal drops to near zero, the artery has collapsed and the applied pressure is approximately equal to the SBP. Thus, the lowest pressure that leads to collapse of the artery is equal to the systolic pressure (SBP). It should be recognized by those of ordinary skill in the art that the applied pressure may be greater than or equal to the systolic pressure and thus, the processor may include an algorithm accounting for this potential discrepancy. The processor may also use the multiple pressure reading/photo sensor signal data pairs to determine the mean arterial blood pressure, by curve fitting the data to determine when the oscillations peak.

The glove includes a display 230 that is electrically coupled to the processor 220. The display 230 indicates the arterial blood pressure and includes an indicator, either auditory, visual or both, for the caregiver to apply more or less pressure so that the processor can determine the transition between the artery being pulsatile and non-pulsatile. The glove can include a start button 250 that will initialize the glove. When the start button is pressed by the care giver the photo sensor and pressure sensor become active. The processor then continually senses the oscillation and applied pressure.

It should be recognized by one of ordinary skill that each finger of the gloved embodiment may have separate sensor units, or only one finger may have a sensor units. In addition, the light source may include a single LED or multiple LEDs and the photo sensor may be a single sensor or an array of sensors. To address the likely signal artifacts caused by a photo sensor pressed against a subject, and to make measurements rapidly, a multiple photo sensor unit design is provided where different parallel photo sensor units are placed on mounts of differing compliance, leading to a range of loading pressures assayed for any given pressure applied by the caregiver. In accordance with embodiments of the present invention, a patient's ABP is estimated with the touch of a caregiver's instrumented glove or probe. This modality is attractive to providers in a range of hazardous or poorly controlled prehospital environments: battlefields, collapsed buildings, motor vehicle accidents with casualties pinned inside, etc.

In certain embodiments, when the pressure is sensed and a pulsatile signal is detected, the blood pressure measuring device will become active. The processor may continually monitor both sensors and only become fully active when both pressure is sensed and a pulsatile signal is detected.

The processor can include logic that will identify false readings. For example, if the pressure measured by the pressure sensor drops to zero and the pulsatile signal is minimal, the processor will assume that the caregiver has removed the glove or wand from the patient. However, if a pulsatile signal exists at a given external pressure and the pressure is further increased and the pulsatile signal is lost then the processor will assume that the measurement is accurate. False readings may also be detected by using an accelerometer to determine if the device is being moved substantially during measurement. Additionally, false readings can be identified by measuring noise in the first sensor. For example, pressure changes, such as large magnitude changes or high frequency changes not consistent with controlled pressing/releasing by a caregiver nor by underlying arterial pulsations which indicate that the device is not being pressed against the patient in a mechanically controlled fashion would be indicative of a false reading.

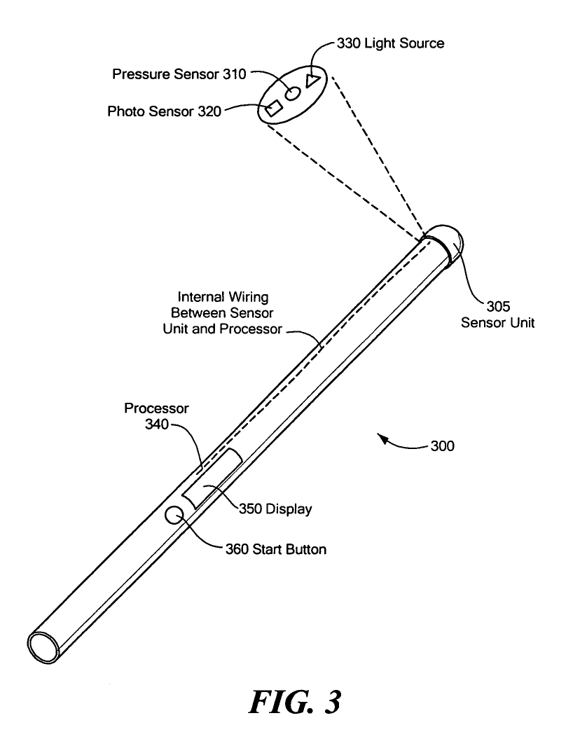

FIG. 3 shows another embodiment in which a first sensor, such as a pressure sensor 310, a second sensor, such as a photo sensor 320, and light source 330 are positioned in a sensor unit 305 on the tip of a wand/probe 300. The sensor unit 305 may be formed on a flat or curvilinear surface. The wand 300 also includes a processor 340 programmed to determine the ABP of the patient. The wand 300 may include an internal or externally connected display 350 for showing the ABP of the patient. The wand and glove units function similarly where the caregiver applies pressure that is sensed by the pressure sensor and the second sensor, such as a photo sensor measures the oscillations of the artery. The amplitude of the oscillations occurs in a frequency range that is consistent with the beating of the heart. In alternative embodiments, a morphology analysis can be performed on the PPG signal/photo sensor signal to identify oscillations whose shape is consistent with a pulse beat. The wand embodiment like the glove can include a start button 360 for beginning the blood pressure monitoring. It should be understood by one of ordinary skill in the art that details and features described for the glove embodiment may be included in the wand embodiment as well as other form factors.

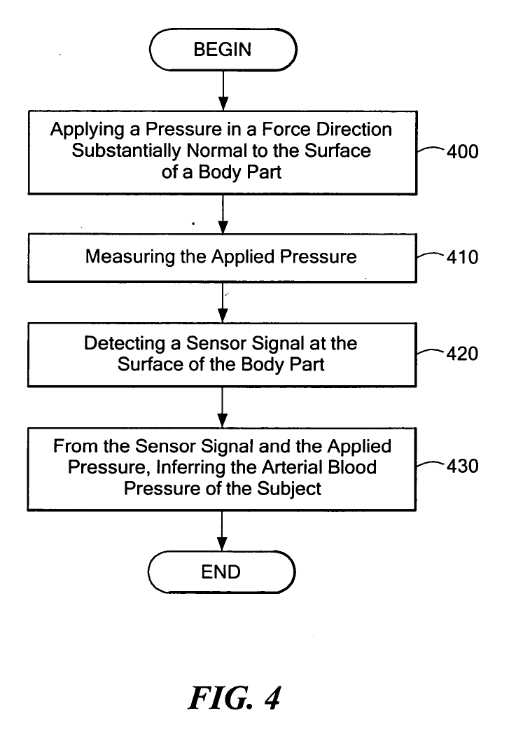

The methodology for obtaining an arterial blood pressure measurement is shown in the flow chart of FIG. 4. First, a caregiver applies a pressure in a force direction substantially normal to the surface of a body part of a subject (400). The applied pressure is measured using a pressure sensor (410). A sensor signal is detected at the surface of the body part (420). For example, the sensor signal may be a PPG sensor signal or a sensor signal from the pressure sensor. If a reflectance photoplethysmogram (PPG) is used, the PPG measures the magnitude of vascular pulsations resulting from different external pressures. From the sensor signal and the applied pressure, the arterial blood pressure can be inferred (430). When the oscillations within the sensor signal fall substantially to zero while pressure is increased by the caregiver, the pressure sensed by the pressure sensor is substantially equal to the SBP. Similarly, the mean arterial pressure can be estimated from the same data obtained from the pressure signal and the sensor signal wherein known mathematical techniques are employed to determine when the oscillations are near a maximum. Further, the diastolic pressure may be determined by analyzing the data using known techniques.

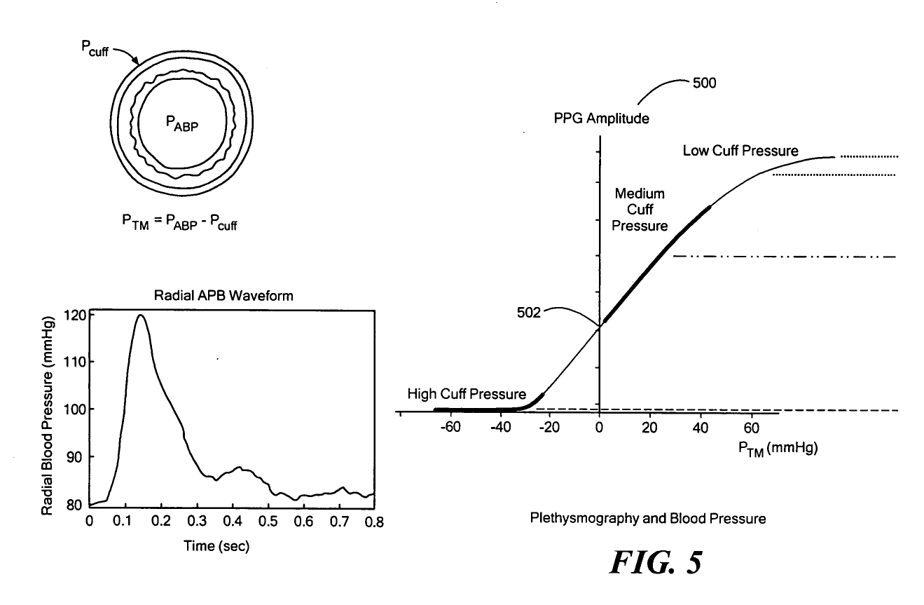

The sigmoid-shaped compliance curve 100 of FIG. 5 demonstrates how the PPG output is a function of the pressure difference across a vascular wall, the transmural pressure (P_{TM}). Note that the slope of the sigmoid-shaped compliance curve 100 is maximal near zero transmural pressure (P_{TM}) 102. This relationship is the basis for measuring the mean arterial pressure with an external pressure applied by a cuff. At maximum compliance, the known external pressure applied by the cuff is equal to the MAP. Thus, with a PPG sensor and the cuff, the MAP can be found by changing the external pressure applied to the extremity and locating the maximum PPG amplitude. Similarly, the compliance curve can be identified by applying a constant external pressure and varying the applied hydrostatic pressure. The maximum pulsations as sensed by a PPG sensor can be determined by taking a number of readings at different external pressure values and fitting the data to a curve to determine the pressure associated with a maximum PPG signal. The processor in both the wand and glove embodiments as described above can be configured to make this determination.

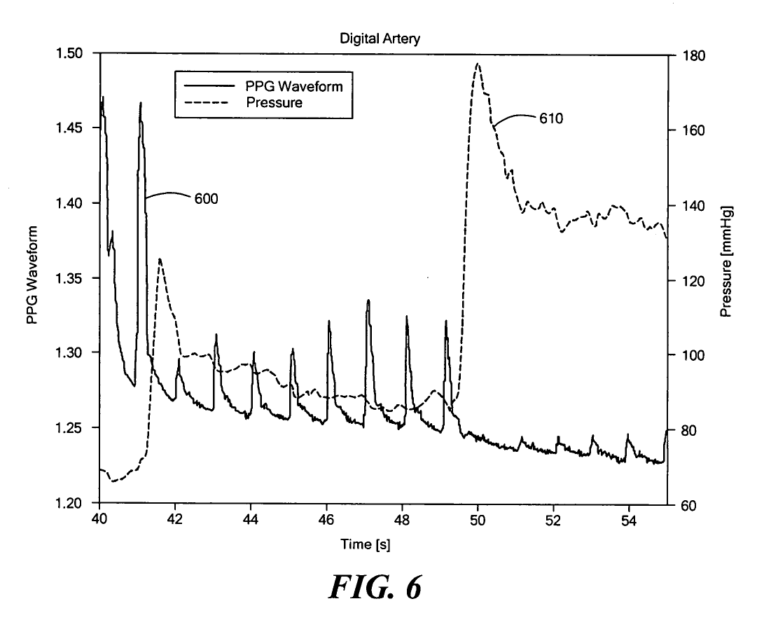

FIG. 6 graphically depicts experimental data obtained using an embodiment of the invention for determining the SBP. A transducer was used to measure external pressure applied necessary to collapse a digital artery. A plethysmograph may be used to determine whether the underlying artery is still pulsatile (i.e., patent) or whether the externally applied pressure has caused it to collapse. In certain embodiments of the invention, pressure sensors alone may serve to detect this transition. However, a photoplethysomgraph has proven more reliable indicator of arterial pulsations than a pressure sensor. In FIG. 6, the PPG signal 600 is plotted, and is used to establish whether or not the artery was pulsating. Overlying the PPG is the magnitude of the pressure applied by the operator (dashed line) 610. The results shown in FIG. 6 may be grouped into three illustrative regions.

I. Between time=40s and time=49s, the external pressure was approximately ˜80 mmHg and the PPG amplitude ˜0.07 V; this is consistent with a patent, pulsating artery.

II. Between time=49s and time=51s, the operator applied much more pressure, increasing to ˜180 mmHg, and the pulsatile quality of the PPG signal disappeared.

III. After time=51s, the operator pressure dropped below 140 mmHg and a small pulsatile PPG signal recurred, indicating that the artery was no longer fully collapsed. Taken together, these data indicate that the peak pressure (SBP) was between 140 mmHg and 160 mmHg. Note that this accuracy is comparable to standard blood pressure cuffs with a reported ±20% measurement error. This blood pressure measurement only required 15 seconds of an operator placing the probe against the subject's finger. In an alternative embodiment, the processor may treat this measurement as ‘unconfirmed’ or ‘preliminary’ because the loss of the pulse only lasted a single beat—this might be caused by the patient's heart skipping a beat rather than the device collapsing the artery. In this embodiment, the device may require the caregiver to either repeat the measurement, or to at least have each pressure/pulse measurement persist for several heart beat's worth of time. The device may communicate the ‘unconfirmed’ or ‘preliminary’ measurement to the display. The device may communicate that the measurement is ‘unconfirmed’ or ‘preliminary’ with some visual or acoustic cue, and offer feedback in some form which informs the caregiver about the necessary action(s) for the measurement confirmation (e.g. pressing harder again, holding pressure longer, etc.)

In accordance with alternate embodiments of the invention, the device may also include a feedback processor to ensure that the operator is pressing directly over the artery, including software, which performs signal quality assessment of the pulsatile signal, and the means to communicate this via visual or audio prompts to the operator. In other embodiments, a grid of pressure or PPG sensors serve to ensure that the device is properly positioned and this is communicated to the operator via visual or audio prompts. In further embodiments, an LED array can be used to determine proper positioning. Each LED may light up sequentially. The processor senses the strength of the signal of each LED and based upon the strength of the sensed signals, the location of the underlying artery can be inferred relative to the sensor array. Based on this data, the device produces a visual/auditory cue for repositioning of the device.

In yet other embodiments of the invention, the operator is instructed to press harder or not as hard, in order to ascertain the pressure at which the artery collapses. A sensor coupled with a processor may detect and communicate to the operator if there is excessive motion artifact or if he/she is making changes to the applied pressure too abruptly or too slowly. Additionally, the processor may automatically analyze the pulsatile signal (pressure and/or PPG sensor data) and the baseline pressure measurement, for automatically computing the SBP, and the means to communicate this to the operator. In an alternative embodiment, the device is capable of automatically modulating the applied pressure by changing the compliance of the force transfer element.

Motion artifacts may arise due to the human operator pressing the sensor through a wide range of applied pressures. Therefore, multiple parallel PPG sensor units may be used, each attached to “springs” of differing compliance, can make human actuation simpler to execute. Specifically, for every force level applied by the human operator, multiple force levels will result at individual pressure and/or PPG sensor units, which may be mounted, if more than one sensor unit is used, on bases of differing compliance. With relatively minimal human manipulation, the pulsatile amplitude for a wide range of external pressures can be queried. As in oscillometry, the force level that produces the maximum oscillation amplitude will indicate mean arterial pressure (because the maximum oscillation would indicate the maximum vascular compliance, which would indicate that the transmural pressure equals zero, which indicates that the external pressure must be equal to the mean arterial pressure).

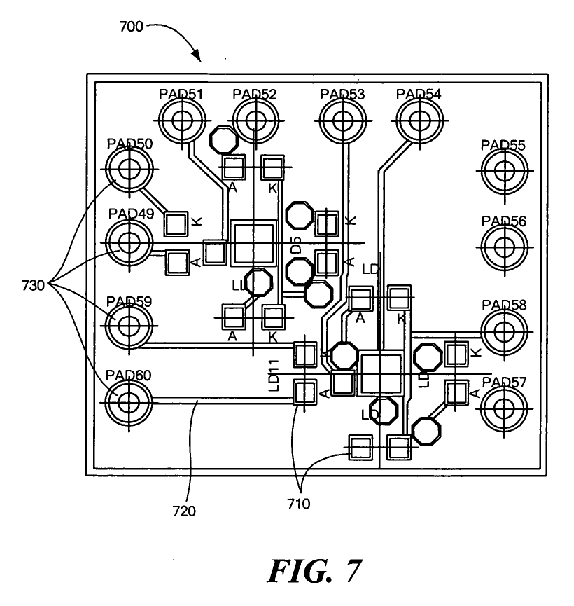

One configuration of photo sensors 700 and LEDs is shown in FIG. 7. The LED's are represented as small squares 710, the two photo sensors (PSs) are represented as the larger squares 720. The circles are the solder pits for input/output to the LED board 730. In other embodiments, the LED/PS units may be mounted separately, with bases of different elasticity, to effect different external pressures when pressed by a caregiver. For example, fingers in the glove embodiment could each have a different LED/PS unit with a base having a different elasticity.

The current blood pressure measuring device may prove challenging in out-of-hospital environments for caregivers, since the blood pressure device may not be positioned at the height of the patient's heart. By convention, ABP is measured at the vertical level of the heart; otherwise, there is a hydrostatic pressure offset (caused by the “column” of blood between the reference location and the actual measurement location). One solution for accounting for the hydrostatic pressure offset is the inclusion of a long fluid-filled column attached to the blood pressure measurement device, the top of which is located at the level of the heart. A pressure gauge at the bottom of this column can directly measure the hydrostatic pressure caused by vertical displacement of the sensor relative to the heart (the top of the column). In other embodiments accelerometers can be used to measure the orientation of the upper and lower arm, from which the height of the arm can be estimated by the processor, and the magnitude of the hydrostatic effect can be estimated and accounted for in the blood pressure reading.

In still other variations for accounting for the hydrostatic offset, a keypad may be included in either the glove or wand embodiments to enable users to key in the height offset, wherein the processor uses the standard formula ρgh to account for the hydrostatic offset. Alternatively, buttons may be used to add/subtract from the offset term (default is zero offset). For instance, an ‘increment’ button may add +10 inches to the offset term, and a user interface enabling the user to accomplish this quickly and accurately is within the scope of this invention.

Other embodiments may include an accelerometer within the glove/wand so that the patient can, when prompted by the device, ‘point’ and the angle of ‘point’ will be used to estimate the magnitude of the offset height. Alternative embodiments may include a rolled-up fluid-filled column integral to the device which can be deployed/unrolled like a tape measure. The tip of the column can be brought to the level of the heart and the device can directly measure the hydrostatic effect caused by the vertical offset between the device's location and the heart, and after this measurement the thin column will automatically roll back up into its storage location, again like a tape measure.

Embodiments of the present invention as herein described may be of particular advantage in prehospital emergency settings. For instance, this device may advantageously permit the measurement of blood pressure in a car crash even before the casualties are extricated. Also, this device would enable the rapid measurement of blood pressure in civilian multi-casualty incidents (e.g. bus crash or train crash). This device may also be advantageous for combat casualty care and to enhance aid kits in settings like commercial flights, or for dangerous expeditions. It may advantageously provide a very desirable method of monitoring blood pressure at home for patients with long-standing high blood pressure. Finally, the device may be advantageous in conventional healthcare settings when expeditious ABP measurement is valuable, such as for an over-crowded Emergency Department, or an Emergency Department after a local disaster, trying to deal with large numbers of casualties.

The described embodiments of the invention are intended to be merely exemplary and numerous variations and modifications are intended to be within the scope of the present invention as described herein and as defined in any appended claims.

We claim:

1. An apparatus for measuring arterial blood pressure in a subject, the apparatus comprising:
a. a force transfer element for applying unidirectional force to a body part of the subject;
b. a first sensor detecting a signal in response to the unidirectional force;
c. a second sensor detecting a signal associated with blood volume within a blood vessel of the body part; and
d. a controller for inferring arterial blood pressure from the sensor signals.

2. An apparatus according to claim 1, wherein the first sensor generates the signal based on mechanical reaction against a caregiver.

3. An apparatus according to claim 1, wherein the first sensor is a force sensor.

4. An apparatus according to claim 3, wherein the force sensor is a pressure sensor.

5. An apparatus according to claim 3, wherein the force sensor serves as the second sensor.

6. An apparatus according to claim 1, wherein the second sensor is a plethsmograph.

7. An apparatus in accordance with claim 6, wherein the plethsmograph is a photoplethysomgraph.

8. An apparatus in accordance with claim 1, wherein the force transfer element is a glove.

9. An apparatus according to claim 1, wherein the force transfer element is characterized by a plurality of pressure regions each having a distinctive compliance.

10. An apparatus according to claim 1, wherein the force transfer element is a wand.

11. An apparatus according to claim 3, having a plurality of force sensors.

12. An apparatus according to claim 11, wherein each force sensor is coupled to a mount having a different compliance characteristic.

13. An apparatus according to claim 1, wherein the signal associated the second sensor measures oscillations of the artery.

14. An apparatus according to claim 13, wherein the controller determines the arterial blood pressure based in part upon the oscillations measured by the second sensor.

15. An apparatus according to claim 1, wherein systolic blood pressure is determined by the controller based in part on the signal of the second sensor.

16. An apparatus according to claim 1 wherein mean arterial blood pressure is determined by the controller based in part on the signal of the second sensor.

17. An apparatus according to claim 1, wherein diastolic blood pressure is determined by the controller based in part on the signal of the second sensor.

18. An apparatus according to claim 1, wherein the controller produces feedback for the caregiver.

19. An apparatus according to claim 18, wherein the feedback indicates repositioning of the apparatus relative to the artery.

20. An apparatus according to claim 18, wherein the feedback indicates that a caregiver should change the applied unidirectional force.

21. An apparatus according to claim 1, wherein the controller includes a user input for accounting for hydrostatic pressure.

22. A method for measuring arterial blood pressure in a subject, the method comprising:
a. applying a pressure in a force direction substantially normal to the surface of a body part of the subject;
b. measuring the pressure applied;
b. detecting a sensor signal at the surface of the body part;
c. inferring arterial blood pressure from the sensor signal and the applied pressure.

23. A method according to claim 22, wherein the sensor signal detects oscillations of the artery.

24. A method according to claim 22, wherein systolic blood pressure is determined based at least on the sensor signal.

25. A method according to claim 14 wherein the applied pressure is measured by a pressure sensor.

26. A method according to claim 22 wherein inferring arterial blood pressure occurs in a controller.

27. A method according to claim 22 wherein the sensor signal is sensed by a photoplethysomgraph.

28. A method according to claim 22 further comprising:
placing a glove on a hand of the caregiver wherein the glove includes a pressure sensor for measuring the applied pressure, a photo sensor for detecting the sensor signal, and a light source.

29. A method according to claim 22, wherein pressure is applied in a force direction substantially normal to the body part of the subject by a wand and wherein the wand includes a sensor unit having a light source, a photo sensor, and a pressure sensor.

30. A method according to claim 22, further comprising:
measuring hydrostatic pressure;
wherein the arterial blood pressure is also inferred based on the hydrostatic pressure.

31. A method according to claim 22 wherein applying a pressure in a direction normal to a body part results from a force transfer element; and:
based in part on the detected sensor signal providing feedback to a caregiver to reposition the force transfer member.
